(19) United States
(12) Patent Application Publication
(10) Pub. No.: US 2017/0340241 A1
(43) Pub. Date: Nov. 30, 2017

(54) ENDOSCOPIC EXAMINATION SUPPORT DEVICE, ENDOSCOPIC EXAMINATION SUPPORT METHOD, AND ENDOSCOPIC EXAMINATION SUPPORT PROGRAM

(71) Applicant: FUJIFILM Corporation, Tokyo (JP)

(72) Inventor: Kenta YAMADA, Tokyo (JP)

(73) Assignee: FUJIFILM Corporation, Tokyo (JP)

(21) Appl. No.: 15/680,858

(22) Filed: Aug. 18, 2017

Related U.S. Application Data
(63) Continuation of application No. PCT/JP2016/001163, filed on Mar. 3, 2016.

(30) Foreign Application Priority Data
Mar. 25, 2015 (JP) 2015-062105

(51) Int. Cl. A61B 5/06; A61B 1/267; A61B 1/04

(52) U.S. Cl. CPC A61B 5/066; A61B 1/04; A61B 1/2676

(57) ABSTRACT

A bronchial image generation unit generates a bronchial image and a position information acquisition unit acquires position information of an endoscope in a bronchus. A passage position information acquisition unit acquires passage position information representing a passage position of the endoscope and a passage propriety information acquisition unit acquires passage propriety information representing portions through which the endoscope can be passed and a portion through which the endoscope cannot be passed. A display control unit displays a bronchial image by changing a display state of a portion of the bronchial image through which the endoscope has been passed and a portion of the bronchial image through which the endoscope has not been passed using the passage position information, and changing a display state of portions of the bronchial image through which the endoscope can be passed and cannot be passed using the passage propriety information.

### CROSS REFERENCE TO RELATED APPLICATIONS

The present application is a Continuation of PCT International Application No. PCT/JP2016/001163 filed on Mar. 3, 2016, which claims priority under 35 U.S.C. §119(a) to Japanese Patent Application No. 2015-062105 filed on Mar. 25, 2015. Each of the above applications is hereby expressly incorporated by reference, in its entirety, into the present application.

### BACKGROUND

### Technical Field

The present invention relates to an endoscopic examination support device, an endoscopic examination support method, and an endoscopic examination support program for supporting an endoscopic examination of a tubular structure, such as bronchi, which has a branched structure.

### Description of the Related Art

In recent years, a technique of observing or treating a tubular structure such as the large intestine or bronchi of a patient using an endoscope has been attracting attention. However, an endoscopic image is an image obtained by indicating the inside of a tubular structure in a two-dimensional image whereas it is possible to obtain an image in which the color or the texture of the inside of the tubular structure is clearly expressed using an imaging element such as a charge coupled device (CCD). For this reason, it is difficult to grasp which position within the tubular structure is represented by the endoscopic image. Particularly, a bronchial endoscope has a small diameter and a narrow field, and therefore, it is difficult to make a distal end of the endoscope reach a target position.

A method for generating a virtual endoscopic image, which is similar to an image actually photographed using an endoscope, using a three-dimensional image acquired through tomography in accordance with a modality of a computed tomography (CT) device, a magnetic resonance imaging (MRI) device, or the like has been proposed. This virtual endoscopic image is used as a navigation image for guiding an endoscope to a target position within a tubular structure. However, even in a case where the navigation image is used, in a case of a structure having routes, such as bronchi, which are branched in multi-stages, a skilled technique is required for making a distal end of an endoscope reach a target position within a short period of time. Particularly, in examination of a tubular structure, such as bronchi, which has a branched structure, in some cases, an examination of all branches in which the entirety of the structure is examined is performed. In such an examination of all branches, it requires great effort to thoroughly examine all the routes. In addition, the tubular structure has multiple branches, and therefore, there is also a possibility that an unexamined portion may remain.

For this reason, a method for easily recognizing an unexamined portion by displaying a tubular structure image which is a three-dimensional image of a tubular structure, and identifiably displaying an examined portion and the unexamined portion using an endoscope in the displayed tubular structure image has been proposed (refer to JP2014-50684A). In addition, a method for recording history of routes where a distal end of an endoscope is moved in a navigation image in order to assist identification of accurate routes in a case of inserting the endoscope into bronchi has been proposed (refer to JP2005-522274A). In addition, a method for extracting bronchial image from a three-dimensional image, displaying the bronchial image with different colors for each division divided by branches, and trimming an edge of the virtual endoscopic image, to be displayed, in accordance with the colors of the division at which an endoscope distal end is positioned has been proposed (refer to JP2012-200403A).

In addition, bronchi become thinner toward a terminal. In contrast, the diameter of an endoscope is predetermined. Therefore, there is a portion in bronchi which cannot be examined depending on the diameter of an endoscope to be used. For this reason, a method for displaying bronchi by classifying the bronchi using colors in accordance with the diameter in a bronchial image has been proposed (refer to JP2007-83034A). Furthermore, a method for presenting the kinds of usable endoscopes in accordance with the diameter of a bronchus on a bronchial image has also been proposed (refer to JP2004-89483A).

### SUMMARY

According to the method disclosed in JP2007-83034A, it is possible to easily identify the diameter of a bronchus by observing the three-dimensional image of the bronchus. However, it is impossible to recognize which portion of the bronchus an endoscope in use can or cannot pass through even by viewing the bronchial image displayed through the method disclosed in JP2007-83034A.

In addition, according to the method disclosed in JP2004-89483A, the kinds of usable endoscopes are presented. Therefore, it is possible to easily recognize a portion of bronchi which can be examined using the endoscope in use. However, the method disclosed in JP2004-89483A is a method for presenting the kinds of usable endoscopes in order to select an endoscope before an examination. For this reason, in the method of JP2004-89483A, it is impossible to determine which portion of bronchi an endoscope can pass through during an examination.

The present invention has been made in consideration of the above-described circumstances, and an object of the present invention is to easily recognize a portion through which an endoscope can pass and a portion through which the endoscope cannot pass in a case of performing an examination of a tubular structure such as bronchi by inserting the endoscope into the tubular structure.

An endoscopic examination support device according to the present invention comprises: tubular structure image generation unit for generating a tubular structure image representing a tubular structure having a branched structure of a subject from a three-dimensional image including the tubular structure; position information acquisition unit for acquiring position information of an endoscope inserted into the tubular structure; passage position information acquisition unit for acquiring passage position information representing a passage position of the endoscope in the tubular structure using the position information; passage propriety information acquisition unit for acquiring passage propriety information representing a portion in the tubular structure through which the endoscope can be passed and a portion in the tubular structure through which the endoscope cannot be passed, by comparing the diameter of the endoscope with the diameter of the tubular structure at each position; and display control unit for displaying the tubular structure image on display unit by changing a display state of a portion in the tubular structure image through which the endoscope has been passed and a portion in the tubular structure image through which the endoscope has not been passed using the passage position information, and changing a display state of a portion in the tubular structure image through which the endoscope can be passed and a portion in the tubular structure image through which the endoscope cannot be passed using the passage propriety information.

The expression “changing a display state” means appealing to a visual sense of a person who views the tubular structure image and changing a state of the tubular structure. For example, the expression means changing color, brightness, contrast, opacity, sharpness, and the like of the tubular structure in the tubular structure image.

In the endoscopic examination support device according to the present invention, the display control unit may change a display state of the tubular structure in accordance with the diameter of the tubular structure.

In addition, in the endoscopic examination support device according to the present invention, the change of the display state may be at least one change of color, brightness, contrast, opacity, or sharpness.

In addition, in the endoscopic examination support device according to the present invention, the display control unit may further change the display state of the portion through which the endoscope has been passed or the portion through which the endoscope has not been passed, in cases where there is a branch in the middle of the portion in the tubular structure image, through which the endoscope has been passed, and the endoscope has not been passed through a portion ahead of the branch.

In addition, in the endoscopic examination support device according to the present invention, the change of the display state of the portion in the tubular structure image through which the endoscope has been passed or the portion in the tubular structure image through which the endoscope has not been passed may be performed by providing a mark to the portion through which the endoscope has been passed.

In addition, in the endoscopic examination support device according to the present invention, the passage position information acquisition unit may acquire the passage position information at sampling intervals synchronized with respiration of the subject.

In addition, in the endoscopic examination support device according to the present invention, the passage position information acquisition unit may detect a movement of the subject and correct the passage position information in accordance with the movement.

In addition, in the endoscopic examination support device according to the present invention, the display control unit may change the display state of the portion in the tubular structure image through which the endoscope can be passed or the portion in the tubular structure image through which the endoscope cannot be passed using the passage propriety information for each interbranch division divided by the branched structure in the tubular structure.

An endoscopic examination support method according to the present invention comprises: generating a tubular structure image representing a tubular structure having a branched structure of a subject from a three-dimensional image including the tubular structure; acquiring position information of an endoscope inserted into the tubular structure; acquiring passage position information representing a passage position of the endoscope in the tubular structure using the position information; acquiring passage propriety information representing a portion in the tubular structure through which the endoscope can be passed and a portion in the tubular structure through which the endoscope cannot be passed, by comparing the diameter of the endoscope with the diameter of the tubular structure at each position; and displaying the tubular structure image on display unit by changing a display state of a portion in the tubular structure image through which the endoscope has been passed and a portion in the tubular structure image through which the endoscope has not been passed using the passage position information, and changing a display state of a portion in the tubular structure image through which the endoscope can be passed and a portion in the tubular structure image through which the endoscope cannot be passed using the passage propriety information.

There may be a program for causing a computer to execute the endoscopic examination support method according to the present invention.

According to the present invention, passage position information representing a passage position of an endoscope in a tubular structure is acquired using position information of the endoscope inserted into the tubular structure. In addition, passage propriety information representing a portion in the tubular structure through which the endoscope can be passed and a portion in the tubular structure through which the endoscope cannot be passed is acquired by comparing the diameter of the endoscope with the diameter of the tubular structure at each position. Moreover, a tubular structure image generated from a three-dimensional image is displayed by changing a display state of a portion in the tubular structure image through which the endoscope has been passed and a portion in the tubular structure image through which the endoscope has not been passed using the passage position information and a display state of the portion in the tubular structure image through which the endoscope can be passed and the portion in the tubular structure image through which the endoscope cannot be passed using the passage propriety information. For this reason, it is possible to easily recognize a route through which the endoscope has been passed and a route through which the endoscope has not been passed and to easily recognize the portion in the tubular structure through which the endoscope can be passed and the portion in the tubular structure through which the endoscope cannot be passed, through observing the tubular structure image. Accordingly, it is possible to efficiently examine the tubular structure using the endoscope.

### BRIEF DESCRIPTION OF THE DRAWINGS

FIG. 1 is a hardware configuration diagram showing an outline of a diagnosis support system to which an endoscopic examination support device according to an embodiment of the present invention is applied.

FIG. 2 is a view showing a schematic configuration of the endoscopic examination support device realized by installing an endoscopic examination support program in a computer.

FIG. 3 is a view illustrating matching.

FIG. 4 is a view illustrating acquisition of passage propriety information.

FIG. 5 is a view showing a bronchial image, an actual endoscopic image, and a virtual endoscopic image displayed on a display.

FIG. 6 is a flowchart showing processing performed in the present embodiment.

FIG. 7 is a view showing a bronchial image which is classified by colors in accordance with the diameter of a bronchus.

FIG. 8 is a view showing a bronchial image in which a display state of a route in the bronchial image through which an endoscope distal end has been passed is further changed, in cases where there is a branch in the middle of the route, through which the endoscope distal end has been passed, and a portion ahead of the branch is a portion through which the endoscope has not been passed.

### DETAILED DESCRIPTION

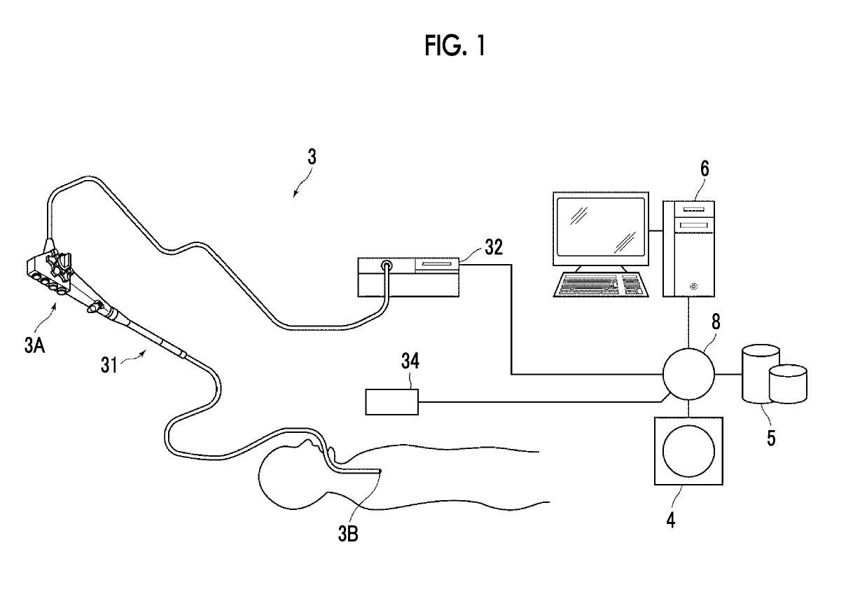

Hereinafter, an embodiment of the present invention will be described with reference to the drawings. FIG. 1 is a hardware configuration diagram showing an outline of a diagnosis support system to which an endoscopic examination support device according to an embodiment of the present invention is applied. As shown in FIG. 1, an endoscope device 3, a three-dimensional image photographing device 4, an image storage server 5, and an endoscopic examination support device 6 are connected to each other in a communicable state via a network 8 in this system.

The endoscope device 3 includes an endoscopic scope 31 imaging the inside of a tubular structure of a subject, a processor device 32 generating an image of the inside of the tubular structure based on a signal obtained through imaging, a position detection device 34 detecting the position and the direction of a distal end of the endoscopic scope 31, and the like.

The endoscopic scope 31 is an endoscopic scope in which an insertion portion inserted into a tubular structure of a subject is connected and attached to an operation portion 3A. The endoscopic scope is connected to the processor device 32 via a universal cord which is detachably connected to the processor device 32. The operation portion 3A includes various buttons for instructing an operation such that a distal end 3B of the insertion portion is curved in the vertical direction and the horizontal direction within a predetermined angular range or for collecting a sample of tissue by operating a puncture needle attached to a distal end of the endoscopic scope 31. In the present embodiment, the endoscopic scope 31 is a flexible mirror for bronchi and is inserted into a bronchus of a subject. Then, light guided by an optical fiber from a light source device which is not shown in the drawing and is provided in the processor device 32 is emitted from the distal end 3B of the insertion portion of the endoscopic scope 31, and an image within the bronchus of the subject is obtained using an imaging optical system of the endoscopic scope 31. The distal end 3B of the insertion portion of the endoscopic scope 31 will be referred to as an endoscope distal end 3B in the following description for ease of the description.

The processor device 32 generates an endoscopic image T0 by converting an imaging signal imaged using the endoscopic scope 31 into a digital image signal and by correcting the quality of the image through digital signal processing such as white balance adjustment and shading correction. The generated image is a moving image represented, for example, by a predetermined sampling rate such as 30 fps. The endoscopic image T0 is transmitted to the image storage server 5 or the endoscopic examination support device 6. Here, in the following description, the endoscopic image T0 photographed using the endoscope device 3 is referred to as an actual endoscopic image T0 in order to distinguish it from a virtual endoscopic image to be described below.

The position detection device 34 detects the position and the direction of the endoscope distal end 3B in the body of the subject. Specifically, the relative position and direction of the endoscope distal end 3B in the body of the subject are detected by detecting the characteristic shape of the endoscope distal end 3B using an echo device having a detection region of a three-dimensional coordinate system in which the position of a specific site of the subject is used as a reference, and the information of the detected position and direction of the endoscope distal end 3B is output to the endoscopic examination support device 6 as position information Q0 (for example, refer to JP2006-61274A). The detected position and direction of the endoscope distal end 3B respectively correspond to a viewpoint and a viewpoint direction of an endoscopic image obtained through imaging. Here, the position of the endoscope distal end 3B is represented by three-dimensional coordinates in which the above-described position of a specific site of the subject is used as a reference. In the following description, the information of the position and the direction is simply referred to as position information. In addition, the position information Q0 is output to the endoscopic examination support device 6 using the same sampling rate as that of the actual endoscopic image T0.

The three-dimensional image photographing device 4 is a device generating a three-dimensional image V0 representing an examination target site of the subject by imaging the site, and specific examples thereof include a CT device, an Mill device, a positron emission tomography (PET) device, and an ultrasound diagnostic apparatus. The three-dimensional image V0 generated by this three-dimensional image photographing device 4 is transmitted to and stored in the image storage server 5. In the present embodiment, the three-dimensional image photographing device 4 generates the three-dimensional image V0 obtained by imaging the chest including bronchi.

The image storage server 5 is a computer storing and managing various kinds of data and includes a large-capacity external storage device and software for managing a database. The image storage server 5 communicates with other devices via the network 8 to transmit and receive image data or the like. Specifically, image data pieces such as the actual endoscopic image T0 acquired by the endoscope device 3 and the three-dimensional image V0 generated by the three-dimensional image photographing device 4 are acquired via the network and are stored in and managed by a recording medium such as the large-capacity external storage device. The actual endoscopic image T0 becomes moving image data imaged in accordance with the movement of the endoscope distal end 3B. For this reason, the actual endoscopic image T0 is preferably transmitted to the endoscopic examination support device 6 without passing through the image storage server 5. The storage format of image data or the communication between the devices via the network 8 is based on protocols such as digital imaging and communication in medicine (DICOM).

The endoscopic examination support device 6 is prepared by installing the endoscopic examination support program of the present invention in a computer. The computer may be a workstation or a personal computer which is directly operated by a doctor performing a diagnosis, or may be a server computer which is connected to the workstation or the personal computer via a network. The endoscopic examination support program is distributed by being recorded in a recording medium such as a digital versatile disc (DVD) or a compact disk read only memory (CD-ROM) and is installed in a computer from the recording medium. Alternatively, the endoscopic examination support program is installed by being stored in a storage device of a server computer connected to a network or in network storage in an accessible state from the outside and by being downloaded in the computer used by a doctor who is a user of the endoscopic examination support device 6 as necessary.

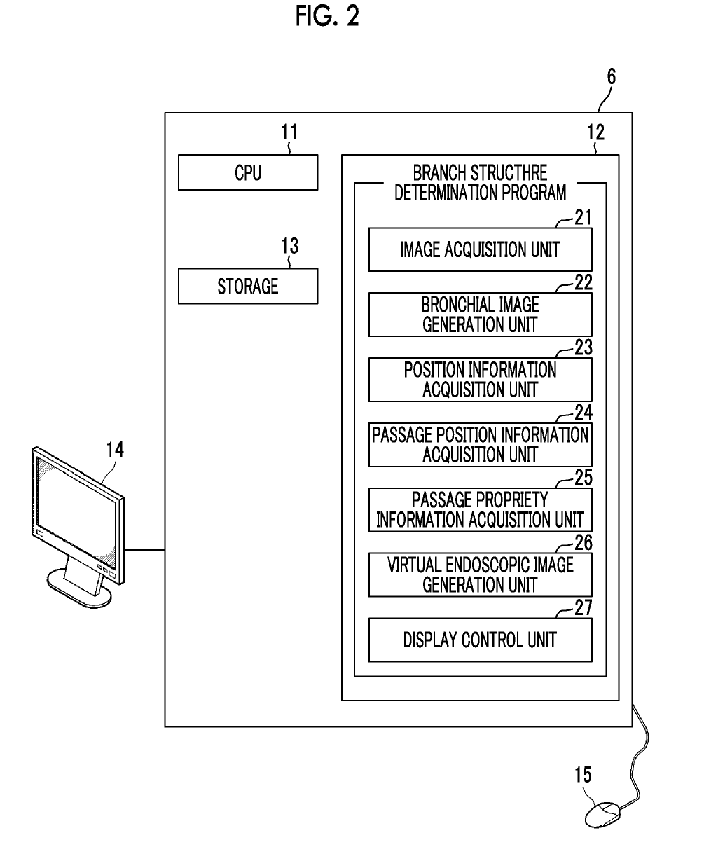

FIG. 2 is a view showing a schematic configuration of the endoscopic examination support device realized by installing the endoscopic examination support program in the computer. As shown in FIG. 2, the endoscopic examination support device 6 includes a central processing unit (CPU) 11, a memory 12, and a storage 13 as a standard workstation configuration. In addition, a display 14 and an input unit 15 such as a mouse are connected to the endoscopic examination support device 6.

The actual endoscopic image T0 and the three-dimensional image V0 acquired from the endoscope device 3, the three-dimensional image photographing device 4, the image storage server 5, and the like via the network 8 and images, information, and the like generated through processing performed in the endoscopic examination support device 6 are stored in the storage 13.

In addition, the endoscopic examination support program is stored in the memory 12. As processing to be executed by the CPU 11, the endoscopic examination support program defines: image acquisition processing for acquiring image data pieces such as the actual endoscopic image T0 generated by the processor device 32 and the three-dimensional image V0 generated in the three-dimensional image photographing device 4; bronchial image generation processing for generating the three-dimensional bronchial image B0 representing a bronchial graph structure from the three-dimensional image V0; position information acquisition processing for acquiring position information of the endoscope distal end 3B inserted into a bronchus; passage position information acquisition processing for acquiring passage position information representing the passage position of the endoscope distal end 3B in bronchi using the position information; passage propriety information acquisition processing for acquiring passage propriety information representing a portion in bronchi through which an endoscope can be passed and a portion in bronchi through which the endoscope cannot be passed, by comparing the diameter of the endoscope distal end 3B with the diameter of a bronchus at each position; virtual endoscopic image generation processing for generating a virtual endoscopic image from the three-dimensional image V0; and display control processing for displaying the bronchial image B0 on the display 14 by changing a display state of a portion in a tubular structure image through which the endoscope has been passed and a portion in the tubular structure image through which the endoscope has not been passed using the passage position information, and changing a display state of a portion in the bronchial image B0 through which the endoscope can be passed and a portion in the bronchial image B0 through which the endoscope cannot be passed using the passage propriety information.

In a case where the CPU 11 performs these kinds of processing in accordance with the program, the computer functions as an image acquisition unit 21, a bronchial image generation unit 22, a position information acquisition unit 23, a passage position information acquisition unit 24, a passage propriety information acquisition unit 25, a virtual endoscopic image generation unit 26, and a display control unit 27. The endoscopic examination support device 6 may includes a plurality of processors performing the image acquisition processing, the bronchial image generation processing, the position information acquisition processing, the passage position information acquisition processing, the passage propriety information acquisition processing, the virtual endoscopic image generation processing, and the display control processing. Here, the bronchial image generation unit 22 corresponds to tubular structure image generation unit.

The image acquisition unit 21 acquires the actual endoscopic image T0 and the three-dimensional image V0 obtained by imaging the inside of a bronchus at a predetermined viewpoint position using the endoscope device 3. The image acquisition unit 21 may acquire the actual endoscopic image T0 and the three-dimensional image V0 from the storage 13 in a case where the images are already stored in the storage 13. The actual endoscopic image T0 is an image representing the inner surface of a bronchus, that is, the inner wall of a bronchus. The actual endoscopic image T0 is displayed on the display 14 by being output to the display control unit 27.

The bronchial image generation unit 22 generates the three-dimensional bronchial image B0 by extracting a structure of bronchi from the three-dimensional image V0. Specifically, the bronchial image generation unit 22 extracts a graph structure of a bronchial region included in the input three-dimensional image V0 as the three-dimensional bronchial image B0, for example, through a method disclosed in JP2010-220742A. Hereinafter, an example of this method for extracting a graph structure will be described.

In the three-dimensional image V0, a pixel in the inside of bronchi corresponds to an air region, and therefore, represented as a region showing a low pixel value. The bronchial wall is represented as a cylindrical or linear structure showing a comparatively high pixel value. The bronchi are extracted through analyzing the structure of the shape based on distribution of pixel values for each pixel.

The bronchi are branched in multi-stages, and the diameter of a bronchus decreases toward a terminal. The bronchial image generation unit 22 detects tubular structures having different sizes so as to detect bronchi having different sizes, by generating a plurality of three-dimensional images having different resolutions by performing multiple resolution conversion on the three-dimensional image V0, and by applying a detection algorithm for each three-dimensional image with each resolution.

First, a Hessian matrix of each pixel of the three-dimensional image at each resolution is calculated and it is determined whether the pixel is within a tubular structure from a magnitude relation of an eigenvalue of the Hessian matrix. The Hessian matrix is a matrix having a second order partial differential coefficient of a density value in each axial (an x-axis, a y-axis, and a z-axis of the three-dimensional image) direction as an element, and becomes 3×3 matrix as shown in the following formula.

$${∇}^{2} I = [ \begin{matrix}{I}_{xx} & {I}_{xy} & {I}_{xz} \\ {I}_{xx} & {I}_{xy} & {I}_{xz} \\ {I}_{xx} & {I}_{xy} & {I}_{xz}\end{matrix} ] {I}_{xx} = \frac{{δ}^{2} I}{δ {x}^{2}} , {I}_{xy} = \frac{{δ}^{2} I}{δ x δ {y}^{2}} , …$$

In a case where eigenvalues of a Hessian matrix at arbitrary pixels are set as λ1, λ2, and λ3, in a case where two eigenvalues among eigenvalues are large and one eigenvalue is close to 0, for example, in a case where λ3, λ2>>λ1, λ1≅0 is satisfied, it is known that the pixels are tubular structures. In addition, an eigenvector corresponding to the minimum eigenvalue (λ1≅0) of the Hessian matrix coincides with a principal axis direction of the tubular structure.

The bronchi can be represented by a graph structure. However, the tubular structures extracted in this manner is not limited to be detected as a graph structure in which all of tubular structures are connected to each other due to an influence of tumor or the like. Whether a plurality of tubular structures are connected to each other is determined by evaluating whether or not each of the extracted tubular structures is within a certain distance and whether or not an angle formed by a principal axis direction of each tubular structure and the direction of a basic line connecting arbitrary points on two extracted tubular structures is within a certain angle, after the detection of the tubular structures from the entirety of the three-dimensional image V0 has been completed. Then, the connection relation of the extracted tubular structures is reconstructed. The extraction of the graph structure of bronchi is completed through the reconstruction.

The bronchial image generation unit 22 can obtain a three-dimensional graph structure representing bronchi as the bronchial image B0 by classifying the extracted graph structure into a start point, an end point, a branch point, and a side and by connecting the start point, the end point, and the branch point by the side. The method for generating the graph structure is not limited to the above-described method, and other methods may be employed.

The position information acquisition unit 23 acquires the position information Q0 detected by the position detection device 34.

The passage position information acquisition unit 24 acquires passage position information Q1 representing the passage position of the endoscope distal end 3B in the bronchi using the position information Q0. For this reason, the passage position information acquisition unit 24 makes a coordinate system of the bronchial image B0 and a coordinate system of the position information Q0 coincide with each other by making the reference point of the coordinate system of the bronchial image B0 and the reference point of the coordinate system of the position information Q0 coincide with each other. Accordingly, it is possible to specify a position corresponding to the position of the endoscope distal end 3B in the bronchial image B0 using the position information Q0. The passage position information acquisition unit 24 acquires three-dimensional coordinates as passage position information Q1 of a position corresponding to the position information Q0 in the bronchial image B0. In a case where the coordinate system of the bronchial image B0 and the coordinate system of the position information Q0 coincide with each other, the passage position information Q1 coincide with the position information Q0. In addition, the passage position information Q1 is acquired using the same sampling rate as that of the position information Q0.

The passage position information Q1 may be acquired at a timing synchronized with respiration of a subject. For example, the passage position information Q1 may be acquired at a timing of expiration or at a timing of inspiration. Accordingly, it is possible to compensate deviation of the position information Q0 caused by respiration. Therefore, it is possible to accurately acquire the passage position information Q1.

In addition, the passage position information Q1 may be corrected in accordance with the movement of the subject by detecting the movement. In this case, a motion sensor for detecting the movement of the subject is prepared, the motion sensor (hereinafter, simply referred to as a sensor) is attached to the chest of the subject, and the movement of the subject is detected using the sensor. The movement of the subject is represented by a three-dimensional vector. In the passage position information acquisition unit 24, the passage position information Q1 acquired based on the position information Q0 may be corrected in accordance with the movement detected by the sensor. The position information Q0 may be corrected in the position detection device 34 in accordance with the movement detected by the sensor. In this case, in the passage position information acquisition unit 24, the passage position information Q1 acquired in accordance with the position information Q0 is corrected by the movement of the subject.

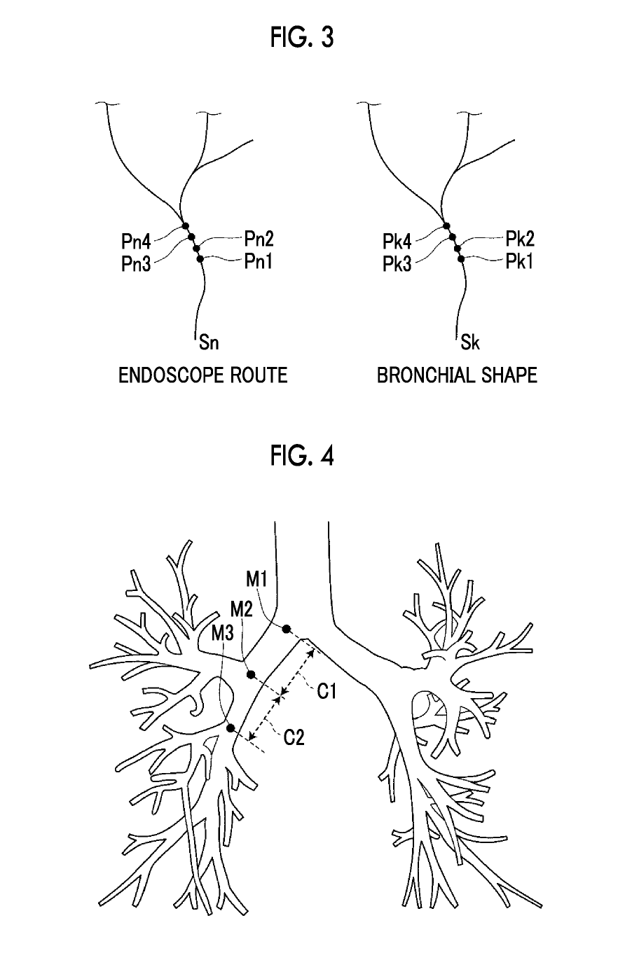

In addition, the passage position information Q1 may be acquired by matching the bronchial image B0 with the actual endoscopic image T0 as disclosed, for example, in JP2013-150650A. Here, the matching is processing for aligning the bronchi represented by the bronchial image B0 and the actual position of the endoscope distal end 3B within the bronchi. For this reason, the passage position information acquisition unit 24 acquires route information of the endoscope distal end 3B within the bronchi. Specifically, a line segment obtained by approximating the position of the endoscope distal end 3B, which has been detected by the position detection device 34, using a spline curve or the like as the route information. As shown in FIG. 3, matching candidate points Pn1, Pn2, Pn3, are set on an endoscope route at sufficiently narrow-range intervals of about 5 mm to 1 cm and matching candidate points Pk1, Pk2, Pk3, . . . are set on a bronchial shape at the same range intervals.

Then, the passage position information acquisition unit 24 performs matching by associating the matching candidate points on the endoscope route with the matching candidate points on the bronchial shape in order from endoscope insertion positions Sn and Sk. Accordingly, it is possible to specify the current position of the endoscope distal end 3B on the bronchial image B0. The passage position information acquisition unit 24 acquires three-dimensional coordinates at the specified position as the passage position information Q1.

The passage propriety information acquisition unit 25 acquires the passage propriety information representing whether or not the endoscope distal end 3B in the bronchi can be passed. Specifically, the passage propriety information acquisition unit acquires passage possibility information Q2 representing that the endoscope distal end 3B can be passed and passage impossibility information Q3 representing that the endoscope distal end 3B cannot be passed. The passage possibility information Q2 and the passage impossibility information Q3 are collectively called passage propriety information. In the present embodiment, the passage propriety information is acquired for each interbranch division which is a division between branch positions of the bronchi.

FIG. 4 is a view illustrating acquisition of passage propriety information. As shown in FIG. 4, the passage propriety information acquisition unit 25 sets branch positions M1, M2, M3, . . . (hereinafter, referred to as Mi) on the bronchial image B0 and sets interbranch divisions C1, C2, C3, . . . (hereinafter, referred to as Cj), for which the passage propriety information is acquired, between two branch positions. The passage propriety information acquisition unit 25 calculates the cross-sectional area of the bronchi at sufficiently narrow-range intervals of about 5 mm to 1 cm in each interbranch division and obtains a cross section having a minimum cross-sectional area. Here, the cross section of the bronchi forms an elliptical shape, and therefore, the passage propriety information acquisition unit 25 obtains a minor axis of the obtained cross section. The passage propriety information acquisition unit 25 sets a bronchial diameter dj of an interbranch division Cj having the obtained target minor axis.

Furthermore, the passage propriety information acquisition unit 25 compares the diameter dl of the endoscope distal end 3B with the bronchial diameter dj of each of the interbranch divisions Cj. In a case where dj>dl is satisfied, the passage propriety information acquisition unit acquires the passage possibility information Q2 indicating that the endoscope distal end 3B can be passed through a target interbranch division Cj. In a case where dj≦dl, the passage propriety information acquisition unit acquires the passage impossibility information Q3 indicating that the endoscope distal end 3B cannot be passed through a target interbranch division Cj.

The passage propriety information acquisition unit 25 acquires passage propriety information with respect to all of the interbranch divisions Cj in the bronchial image B0. The diameter of the bronchi becomes thinner toward a terminal. For this reason, the passage propriety information acquisition unit 25 acquires the passage propriety information from an entrance of a bronchus (that is, a portion close to the mouth of the human body) toward a terminal of the bronchus. In a case where the passage impossibility information Q3 is acquired in a certain interbranch division Cj, the passage impossibility information Q3 may be assigned for interbranch divisions ahead of the certain interbranch division without acquiring the passage propriety information. Accordingly, it is possible to reduce the amount of calculation for acquiring the passage propriety information.

The passage propriety information may be acquired at sufficiently narrow-range intervals of about 5 mm to 1 cm with respect to the entire bronchial image B0 instead of acquiring the passage propriety information for each interbranch division Cj. Even in this case, in a case where the passage impossibility information Q3 indicating that the endoscope distal end cannot be passed at a certain position is acquired after acquiring the passage propriety information from the entrance of the bronchi toward the terminal of the bronchi, the passage impossibility information Q3 may be assigned for bronchi ahead of the certain position.

The virtual endoscopic image generation unit 26 generates a virtual endoscopic image K0, which describes the inner wall of a bronchus and is viewed from a viewpoint of the inside of the three-dimensional image V0 corresponding to the viewpoint of the actual endoscopic image T0, from the three-dimensional image V0. Hereinafter, the generation of the virtual endoscopic image K0 will be described.

The virtual endoscopic image generation unit 26 first acquires a projection image through central projection performed by projecting a three-dimensional image on a plurality of visual lines extending in radial lines from a viewpoint onto a predetermined projection plane while having the position represented by the passage position information Q1 in the bronchial image B0, that is, the position of the endoscope distal end 3B as the viewpoint, using the latest passage position information Q1 acquired by the passage position information acquisition unit 24. This projection image becomes the virtual endoscopic image K0 virtually generated as an image which is photographed at a distal end position of the endoscope. As a specific method of the central projection, it is possible to use, for example, a well-known volume rendering method. In addition, the view angle (the range of the visual lines) of the virtual endoscopic image K0 and the center of the visual field (center in the projection direction) are set in advance through input or the like performed by a user. The generated virtual endoscopic image K0 is output to the display control unit 27.

The display control unit 27 displays the bronchial image B0, the actual endoscopic image T0, and the virtual endoscopic image K0 on the display 14. At this time, the display control unit 27 displays the bronchial image B0 by changing a display state of the position where the endoscope distal end 3B has been passed and the position where the endoscope distal end has not been passed, based on the passage position information Q1. In the present embodiment, the display control unit 27 changes the display state of the position where the endoscope distal end 3B has been passed and the position where the endoscope distal end has not been passed, by displaying a black circle dot at the position where the endoscope distal end 3B has been passed, that is, the position at which the passage position information Q1 has been acquired. A predetermined mark or a pattern may be given to the position where the endoscope distal end 3B has been passed, instead of the dot. In addition, in the bronchial image B0, the color or the pattern of the position where the endoscope distal end 3B has been passed and the position where the endoscope distal end has not been passed may be changed. In addition, at least one of brightness, contrast, opacity, or sharpness of the position where the endoscope distal end 3B has been passed and the position where the endoscope distal end has not been passed may be changed.

In addition, the display control unit 27 displays the bronchial image B0 on the display 14 by changing a display state of a portion in the bronchial image B0 through which the endoscope distal end 3B can be passed and a portion in the bronchial image through which the endoscope distal end cannot be passed, based on the passage propriety information. In the present embodiment, the display control unit 27 displays the bronchial image B0 on the display 14 by changing the color of the portion in the bronchial image B0 through which the endoscope can be passed and the portion in the bronchial image through which the endoscope cannot be passed. The pattern to be given may be changed instead of changing the color thereof. In addition, at least one of brightness, contrast, opacity, or sharpness of the portion through which the endoscope can be passed and the portion through which the endoscope cannot be passed may be changed.

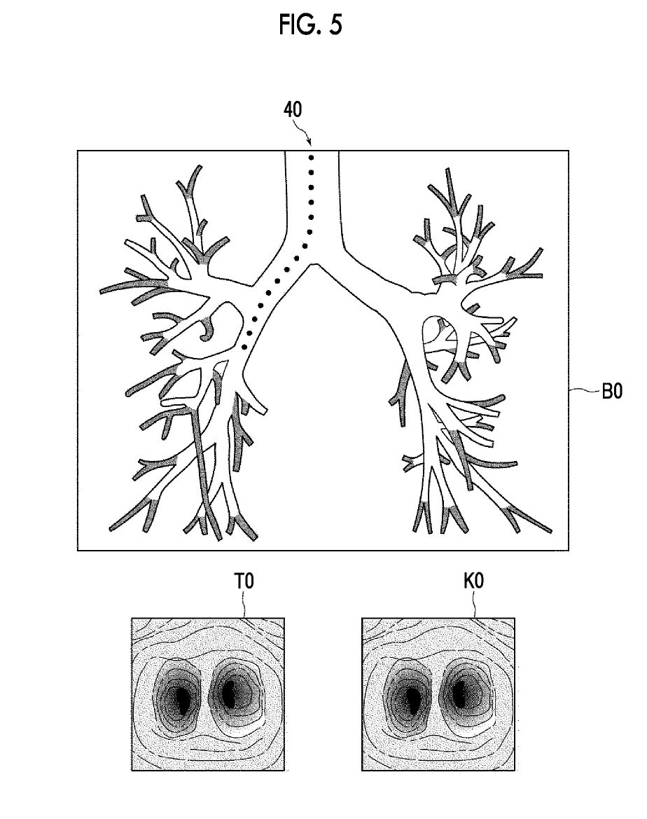

FIG. 5 is a view showing the bronchial image B0, the actual endoscopic image T0, and the virtual endoscopic image K0 displayed on the display 14. As shown in FIG. 5, a plurality of dot-shaped marks 40 which represent the positions where the endoscope distal end 3B has been passed are given to the bronchial image B0. In addition, the color of bronchi through which the endoscope distal end 3B can be passed is regarded as different from the color of bronchi through which the endoscope distal end cannot be passed. FIG. 5 shows a difference between the color of the bronchi through which the endoscope distal end can be passed and the color of the bronchi through which the endoscope distal end cannot be passed, by representing bronchi through which the endoscope distal end cannot be passed using only gray.

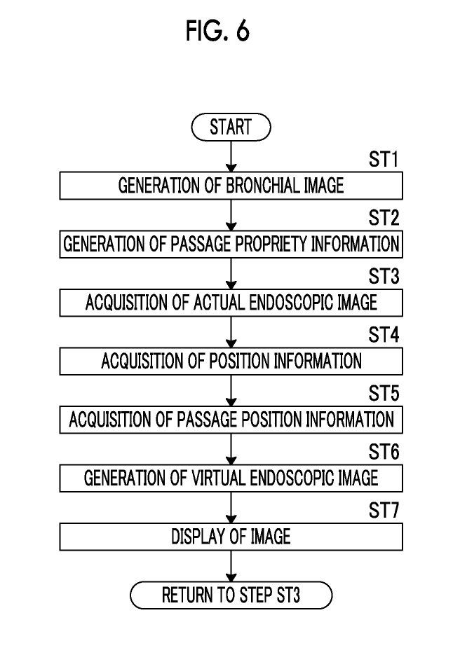

Next, processing performed in the present embodiment will be described. FIG. 6 is a flowchart showing processing performed in the present embodiment. The three-dimensional image V0 is obtained by the image acquisition unit 21 and is stored in the storage 13. First, the bronchial image generation unit 22 generates the bronchial image B0 from the three-dimensional image V0 (Step ST1). The bronchial image B0 may be generated in advance and may be stored in the storage 13. In addition, the passage propriety information acquisition unit 25 acquires passage propriety information representing whether or not the endoscope distal end 3B in the bronchi can be passed (Step ST2). The passage propriety information may be generated in advance and may be stored in the storage 13. In addition, the generation of the bronchial image B0 and the acquisition of the passage propriety information may be performed in parallel or the acquisition of the passage propriety information may be performed prior to the generation of the bronchial image B0.

The image acquisition unit 21 obtains the actual endoscopic image T0 (Step ST3), the position information acquisition unit 23 acquires the position information Q0 detected by the position detection device 34 (Step ST4), the passage position information acquisition unit 24 acquires the passage position information Q1 representing the passage position of the endoscope distal end 3B in the bronchi (Step ST5) using the position information Q0. Next, the virtual endoscopic image generation unit 26 generates the virtual endoscopic image K0, which describes the inner wall of a bronchus and is viewed from a viewpoint of the inside of the three-dimensional image V0 corresponding to a viewpoint of the actual endoscopic image T0, from the three-dimensional image V0 (Step ST6). The display control unit 27 displays the bronchial image B0, the actual endoscopic image T0, and the virtual endoscopic image K0 on the display 14 (image display: Step ST7) and the process returns to Step ST3. In the bronchial image B0 displayed on the display 14, the marks 40 are given to the position where the endoscope distal end 3B has been passed as shown in FIG. 5, and the color of the portion through which the endoscope distal end 3B can be passed and the color of the portion through which the endoscope distal end cannot be passed are changed.

In this manner, in the present embodiment, the bronchial image B0 is displayed by changing a display state of a portion in the bronchial image B0 through which the endoscope distal end 3B has been passed and a portion in the bronchial image through which the endoscope distal end has not been passed using the passage position information Q1, and changing a display state of a portion in the bronchial image B0 through which the endoscope distal end 3B can be passed and a portion in the bronchial image through which the endoscope distal end cannot be passed using the passage propriety information. For this reason, it is possible to easily recognize a route through which the endoscope distal end 3B has been passed and a route through which the endoscope distal end has not been passed and to easily recognize the portion in the bronchi through which the endoscope distal end 3B can be passed and the portion in the bronchi through which the endoscope distal end cannot be passed, through observing the displayed bronchial image B0. Accordingly, it is possible to efficiently examine the bronchi using the endoscope.

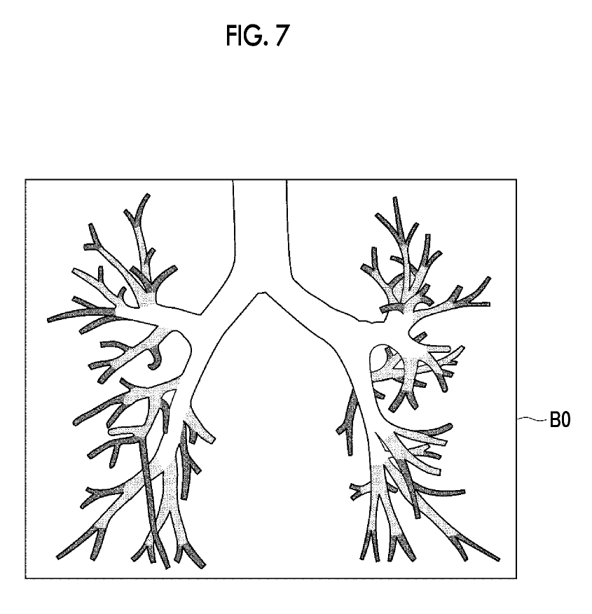

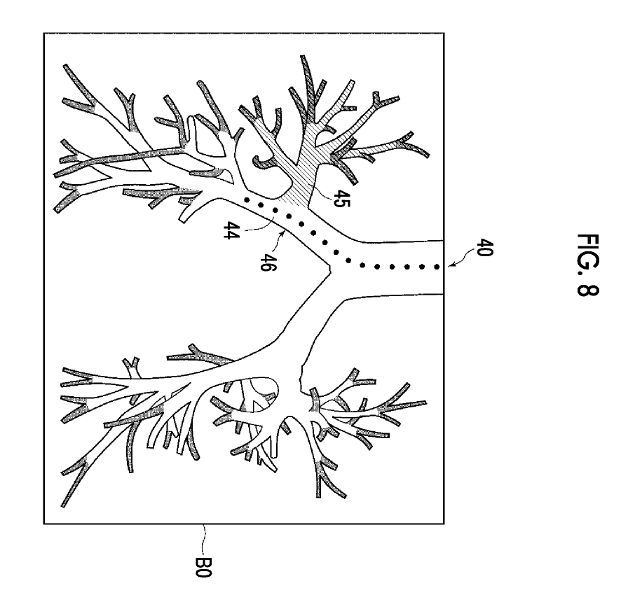

In the above-described embodiment, the display state of the bronchi may be changed in accordance with the diameter of the bronchi in the bronchial image B0. For example, a minor axis of a section having a minimum cross-sectional area may be obtained as the diameter of a bronchus for each of the interbranch divisions and the colors of the interbranch divisions in the bronchial image B0 may vary in accordance with the size of the obtained diameter. In this case, the color of a bronchus is classified as red in a case where the diameter of the bronchus is less than 2 mm, the color of a bronchus is classified as blue in a case where the diameter of the bronchus is 2 mm to 5 mm, and the color of a bronchus is classified as yellow in a case where the diameter of the bronchus is greater than or equal to 5 mm. FIG. 7 is a view showing a bronchial image which is classified by colors in accordance with the diameter of a bronchus. In FIG. 7, the red color is represented by dark gray, the blue color is represented by light gray, and the yellow color is represented by colorlessness. Accordingly, it is possible to easily recognize the diameter of the bronchi in a case where the bronchial image B0 is viewed. In addition, the classification of the diameter of the bronchi using colors is not limited to be three-stage classification, and may be two-stage classification or four- or more-stage classification. In addition, at least one of brightness, contrast, opacity, or sharpness of the bronchi may be changed instead of changing the color in accordance with the diameter of the bronchi.

In addition, in the above-described embodiment, in cases where there is a branch in the middle of a route in the bronchial image B0, through which the endoscope distal end 3B has been passed, and a route ahead of the branch is a route through which the endoscope distal end has not been passed, the display state of the route through which the endoscope distal end has been passed may be further changed. For example, in the bronchial image B0 shown in FIG. 8, the marks 40 are given to the route through which the endoscope distal end 3B has been passed, and the endoscope distal end 3B passes through a branch position 46, at which a bronchus is divided into two bronchi 44 and 45, and advances in the direction of the bronchus 44. In this case, the bronchus 45 enters an unexamined state. For this reason, it is preferable to change the color of the portion of the unexamined bronchus 45 in the bronchial image B0. Here, in FIG. 8, the change of the color of the unexamined portion is indicated by hatching the unexamined portion. Accordingly, it is possible to easily recognize an unexamined bronchus while viewing the bronchial image B0. The color of an examined portion may be changed instead of changing the color of the unexamined portion. In addition, at least one of brightness, contrast, opacity, or sharpness may be changed instead of changing the color thereof.

In addition, in the above-described embodiment, the passage position information Q1 may be acquired by matching the three-dimensional image V0 with the actual endoscopic image T0 in the passage position information acquisition unit 24. In the case of performing such matching, it is impossible to accurately match the three-dimensional image V0 with the actual endoscopic image at a position other than the branch position of the bronchi. For this reason, in the case of matching the three-dimensional image V0 with the actual endoscopic image T0, it is preferable to acquire the passage position information Q1 by performing the matching at only the branch position of the bronchi.

In addition, in the above-described embodiment, the bronchial image B0 is extracted from the three-dimensional image V0 and the virtual endoscopic image K0 is generated using the bronchial image B0. However, the virtual endoscopic image K0 may be generated from the three-dimensional image V0 without extracting the bronchial image B0.

In addition, in the above-described embodiment, the case where the endoscopic examination support device of the present invention is applied for observing the bronchi has been described. However, the present invention is not limited thereto and can be applied even to a case of observing a tubular structure, such as blood vessels, which has a branched structure using an endoscope.

Hereinafter, the effect of the embodiment of the present invention will be described.

It is possible to easily recognize the diameter of a tubular structure by changing a display state of the tubular structure in accordance with the diameter of the tubular structure.

In cases where there is a branch in the middle of a portion in a tubular structure image, through which an endoscope has been passed, and the endoscope has not been passed through a portion ahead of the branch, a display state of the portion through which the endoscope has been passed and a portion through which the endoscope has not been passed may be further changed. Accordingly, it is possible to recognize that the unexamined portion remains. Therefore, it is possible to prevent forgetfulness of an examination.

It is possible to suppress the change in the position of the tubular structure caused by respiration of a subject by acquiring passage position information at sampling intervals synchronized with the respiration. As a result, it is possible to accurately acquire the passage position information.

It is possible to suppress the change in the position of the tubular structure caused by movement of a subject by detecting the movement of the subject and correcting passage position information in accordance with the movement. As a result, it is possible to accurately acquire the passage position information.

A display state of a portion in a tubular structure image through which an endoscope can be passed and a portion in the tubular structure image through which the endoscope cannot be passed may be changed using the passage propriety information for each division divided by a branched structure in the tubular structure. Accordingly, it is possible to recognize whether or not the endoscope can be passed for each division divided by branches.

What is claimed is:

1. An endoscopic examination support device comprising:
tubular structure image generation unit for generating a tubular structure image representing a tubular structure having a branched structure of a subject from a three-dimensional image including the tubular structure;
position information acquisition unit for acquiring position information of an endoscope inserted into the tubular structure;
passage position information acquisition unit for acquiring passage position information representing a passage position of the endoscope in the tubular structure using the position information;
passage propriety information acquisition unit for acquiring passage propriety information representing a portion in the tubular structure through which the endoscope can be passed and a portion in the tubular structure through which the endoscope cannot be passed, by comparing the diameter of the endoscope with the diameter of the tubular structure at each position; and
display control unit for displaying the tubular structure image on display unit by changing a display state of a portion in the tubular structure image through which the endoscope has been passed and a portion in the tubular structure image through which the endoscope has not been passed using the passage position information, and changing a display state of a portion in the tubular structure image through which the endoscope can be passed and a portion in the tubular structure image through which the endoscope cannot be passed using the passage propriety information.

2. The endoscopic examination support device according to claim 1,
wherein the display control unit changes a display state of the tubular structure in accordance with the diameter of the tubular structure.

3. The endoscopic examination support device according to claim 1,
wherein the change of the display state is at least one change of color, brightness, contrast, opacity, or sharpness.

4. The endoscopic examination support device according to claim 1,
wherein the display control unit further changes the display state of the portion through which the endoscope has been passed or the portion through which the endoscope has not been passed, in a case where there is a branch in the middle of a route in the tubular structure image, through which the endoscope has been passed, and the endoscope has not been passed through a portion ahead of the branch.

5. The endoscopic examination support device according to claim 1,
wherein the change of the display state of the portion in the tubular structure image through which the endoscope has been passed or the portion in the tubular structure image through which the endoscope has not been passed is performed by providing a mark to the portion through which the endoscope has been passed.

6. The endoscopic examination support device according to claim 1,
wherein the passage position information acquisition unit acquires the passage position information at sampling intervals synchronized with respiration of the subject.

7. The endoscopic examination support device according to claim 1,
wherein the passage position information acquisition unit detects a movement of the subject and corrects the passage position information in accordance with the movement.

8. The endoscopic examination support device according to claim 1,
wherein the display control unit changes the display state of the portion in the tubular structure image through which the endoscope can be passed or the portion in the tubular structure image through which the endoscope cannot be passed using the passage propriety information for each interbranch division divided by the branched structure in the tubular structure.

9. An endoscopic examination support method comprising:
generating a tubular structure image representing a tubular structure having a branched structure of a subject from a three-dimensional image including the tubular structure;
acquiring position information of an endoscope inserted into the tubular structure;
acquiring passage position information representing a passage position of the endoscope in the tubular structure using the position information;
acquiring passage propriety information representing a portion in the tubular structure through which the endoscope can be passed and a portion in the tubular structure through which the endoscope cannot be passed, by comparing the diameter of the endoscope with the diameter of the tubular structure at each position; and
displaying the tubular structure image on display unit by changing a display state of a portion in the tubular structure image through which the endoscope has been passed and a portion in the tubular structure image through which the endoscope has not been passed using the passage position information, and changing a display state of a portion in the tubular structure image through which the endoscope can be passed and a portion in the tubular structure image through which the endoscope cannot be passed using the passage propriety information.

10. A non-transitory computer-readable recording medium having stored therein an endoscopic examination support program causing a computer to execute:
a step of generating a tubular structure image representing a tubular structure having a branched structure of a subject from a three-dimensional image including the tubular structure;
a step of acquiring position information of an endoscope inserted into the tubular structure;
a step of acquiring passage position information representing a passage position of the endoscope in the tubular structure using the position information;
a step of acquiring passage propriety information representing a portion in the tubular structure through which the endoscope can be passed and a portion in the tubular structure through which the endoscope cannot be passed, by comparing the diameter of the endoscope with the diameter of the tubular structure at each position; and
a step of displaying the tubular structure image on display unit by changing a display state of a portion in the tubular structure image through which the endoscope has been passed and a portion in the tubular structure image through which the endoscope has not been passed using the passage position information, and changing a display state of a portion in the tubular structure image through which the endoscope can be passed and a portion in the tubular structure image through which the endoscope cannot be passed using the passage propriety information.
